(19) United States
(12) Patent Application Publication
(10) Pub. No.: US 2024/0000103 A1
(43) Pub. Date: Jan. 4, 2024

(54) METHOD FOR PRODUCING A PROTEIN MEAL FROM INSECTS, IN PARTICULAR FROM INSECT LARVAE AND FROM INSECT PUPAE, OR FROM WORMS, AND DRYING APPARATUS FOR USE IN SUCH A METHOD

(71) Applicant: BÜHLERINSECT TECHNOLOGY SOLUTIONS AG, Uzwil (CH)

(72) Inventors: Kees Wilhelmus Petrus AARTS, Vught (NL); Tarique ARSIWALLA, Zeist (NL); Maurits Petrus Maria JANSEN, Bavel (NL)

(21) Appl. No.: 18/250,492

(22) PCT Filed: Sep. 20, 2021
(86) PCT No.: PCT/EP2021/075783

§ 371 Date: Apr. 25, 2023

(30) Foreign Application Priority Data
Oct. 28, 2020 (EP) 20204250.3

(51) Int. Cl. A23J 1/02; A23K 20/147; A23K 10/20; A23K 40/00; A23L 33/17; A23J 3/04

(52) U.S. Cl. CPC A23J 1/02; A23K 20/147; A23J 3/04; A23K 40/00; A23L 33/17; A23K 10/20

(57) ABSTRACT

Disclosed is a method for producing a protein meal from insects, in particular from insect larvae and insect pupae, or from worms, comprising the steps of: a) comminuting the insects or worms to form a pulp or purée; b) splitting the pulp or purée into a fat fraction, a solids-containing fraction and an aqueous fraction; c) mixing the aqueous fraction and the solids-containing fraction to form a homogenous compound; d) drying the homogenous compound, with a dried protein meal being obtained. Further disclosed is a drying apparatus for use in such a method, comprising drying devices and a device for comminuting clumps of the homogenous compound.

The present invention relates to a method for producing a protein meal from insects, in particular from insect larvae and from insect pupae, or from worms, according to claim 1 and to a drying apparatus for such a method according to claim 11.

The invention relates to the field of obtaining nutrients, feedstuffs, and foodstuffs from insects or worms. In particular, the invention provides a method for converting insects or worms into nutrient streams comprising a fat fraction, an aqueous fraction and/or a solids-containing fraction. The aqueous fraction and/or the solids-containing fraction contain protein.

In recent decades, interest in the use of insects and worms as food and feed sources has increased—in particular, in view of the growing world population and the increasing demand for alternative and sustainable protein sources for the livestock industry. Since insects and worms are usually rich in proteins and fats, they have a relatively high nutritional or caloric value and are therefore particularly suitable for human nutrition and for livestock breeding.

From an economic and ecological perspective, it is desirable to be able to grow and process insects and worms on an industrial scale in order to produce standardized nutrients that can then be used in the production of food or feed.

GB 2 239 655 A from the unrelated field of processing fish waste discloses a method for obtaining fish meal and fish oil. The fish waste is boiled and then divided into solids and a mixture of water and residual materials. The mixture is heated and then separated by means of centrifuging. The residual water is evaporated to concentrate the solids out of it, which are subsequently dried and ground. The end product is fish meal and fish oil.

A disadvantage of this known method is that it is aimed at processing fish waste and is unsuitable for processing insects and worms because of the cooking step inter alia. In addition, for fish meal and fish oil to be obtained from fish waste, this method has a high energy consumption, which is neither sustainable nor economical.

Several publications disclose that certain nutrients, such as proteins or fats, can be obtained from insects.

For example, CN 104531333 A discloses a method for obtaining fat from certain insect larvae. In this case, aged insect larvae or fresh insect pupae are dried at a constant temperature. The dried compound is ground finely. The ground compound is treated with acetone or gasoline, then residues filtered out and fat is obtained by evaporation.

A disadvantage of this known method is that it too has a high energy requirement and comprises intermediate chemical steps which are unsuitable for obtaining a high-quality protein meal from insects or worms.

It is an object of the present invention to overcome at least one aspect of the disadvantages known from the prior art. In particular, a high-quality protein meal is to be obtained from insects, in particular from insect larvae and from insect pupae, or from worms, wherein the method is also sustainable from the point of view of energy. It is further an object of the present invention to provide a drying apparatus suitable in particular for such a method. The method as well as the drying apparatus should also be suitable for industrial application.

These objects are achieved at least in part by the features of the independent claims. Further advantageous embodiments result from combinations with the features of the corresponding dependent claims and from the statements in the description and figures.

The method according to the invention for producing a protein meal from insects or from worms comprises the following steps:
a) comminuting the insects or worms to form a pulp or purée;
As a result of the comminution, the insects or worms are made available in a form which simplifies further processing and thus the production of the protein meal. The insects or worms should be comminuted finely enough that the fat can be completely removed. However, it has been found to be advantageous if no emulsion is produced during the comminution of the insects or worms since such an emulsion makes the separation of fat significantly more difficult.
b) splitting the pulp or the purée into a fat fraction, a solids-containing fraction and an aqueous fraction;
The solids-containing fraction as well as the aqueous fraction contain proteins.
The aqueous fraction contains soluble components, in particular proteins in soluble form. By splitting the pulp or the purée, the two fractions containing the substantial proportion of the protein are separated and thus enable efficient and economical recovery of the protein present in the insects or worms.
c) mixing the aqueous fraction and the solids-containing fraction to form a homogenous compound;
The fractions each containing a significant amount of proteins are thus combined for further processing.
d) drying the homogenous compound, with a dried protein meal being obtained.
The dried protein meal has a high concentration of proteins and is simple and easy to condition, store, and reuse.
The dried protein meal advantageously has a residual water content of less than 15 wt %, advantageously less than 10 wt %. It has been found that with a residual water content of less than 15 wt %, advantageously less than 10 wt %, the dried protein meal has a shelf life which is significantly longer than for a protein meal with a higher residual water content.

The method according to the invention is characterized by a simple design and continuous production of the protein meal to be produced. Production can be automated and the method can be integrated into a continuous process flow.

This method has proven advantageous in particular for producing a protein meal from insect larvae, e.g., fly larvae or mealworm larvae. The insects, the insect larvae, insect pupae or the worms can also be processed when alive, wherein they are advantageously anesthetized beforehand or at least inactivated.

Optionally, the pulp or the purée can be heated, e.g., during or after the

comminution (step a)), wherein the temperature is advantageously selected such that, for example, any microorganisms present are killed off and enzymes are inactivated. A heating temperature of 70° C. to 125° C., further advantageously from 80° C. to 100° C., has proven advantageous. Particularly advantageous is a heating temperature of 85° C. to 90° C., which is maintained over a specific period of, for example, 5 seconds to 10 minutes. The energy requirement for heating is thus kept low, but the killing off of any bacteria present is ensured.

For example, heating is carried out in a centrifuge, wherein the holding time is 15 seconds at 90° C. The pulp or purée remains in the centrifuge for approximately 45 seconds and has an outlet temperature of approximately 80° C.

Preferably, before step c), the water content of the aqueous fraction is reduced, whereby the homogenous compound obtained by mixing can be dried (in step d)) using less energy and possibly taking less time than in the unreduced state.

The water content of the aqueous fraction is preferably reduced in an evaporator before step c), whereby the water content can be reduced in a simple and energy-efficient manner. The evaporator comprises, for example, an evaporator, in particular a rising film evaporator (also called rising tube or climbing film evaporator), or a falling film evaporator.

As an alternative to an evaporator, a filter can also be used. Using filter processes, the aqueous fraction is concentrated or pre-dewatered and thus the water content of the aqueous fraction is reduced. A membrane filter has proven to be particularly advantageous for filtering.

Preferably, after step d), the dried protein meal is ground, which makes it easy to condition and reuse. The grinding provides the dried protein meal with a large surface area, which has been shown to be advantageous in particular when the protein meal is used in mixtures.

The comminution (step a)) preferably takes place in a cutter or in a press, whereby the resulting pulp or purée has an optimal consistency for further processing.

In the present context, a “cutter” is understood to mean a machine for comminution which is operated continuously and is equipped with a cutting device (e. g., knife).

The comminution of the larvae or worms can also be achieved by means of a press, wherein the solids-containing fraction is separated simultaneously from the fat fraction and/or the aqueous fraction.

The pulp or the purée is preferably split (step b)) in a separator which enables a simple physical/mechanical separation of the fractions. A multi-phase decanter, which is advantageously horizontal, has proven to be particularly advantageous for this purpose.

If the pulp or purée is comminuted by means of a cutter or the like, splitting advantageously takes place in a 3-phase separator so that the fat fraction, the solids-containing fraction and the aqueous fraction are subsequently available for further use.

If the pulp or the purée is comminuted by means of a press or the like, splitting advantageously takes place in a 2-phase separator since the solids-containing fraction is already separated during pressing. The fat fraction and the aqueous fraction are then separated in the 2-phase separator. Subsequently, the fat fraction, the solids-containing fraction and the aqueous fraction are available.

Preferably, clumps of the homogenous compound in step c) and/or in step d) are comminuted (=step e)), as a result of which the dried protein meal has a more uniform particle size.

The clumps can be comminuted, for example, in the mixer before, during or after the mixing process (step c)). For this purpose, the mixer has, for example, a corresponding device, such as, for example, a so-called “clump breaker.”

Alternatively or additionally, the clumps are comminuted, for example, during the drying process (step d)). For this purpose, for example, a corresponding device is provided which can be part of a drying apparatus.

Preferably, the homogenous compound is fed during mixing (step c)) by means of a conveying device for drying by means of a drying apparatus (step d)) in order to avoid an undesired accumulation of the homogenous compound in the drying apparatus since such accumulation can interfere with uniform drying of the homogenous compound. The homogenous compound is advantageously introduced continuously by means of the conveying device.

Alternatively or additionally, the homogenous compound is fed after mixing (step c)) by means of at least one conveying device for drying by means of a drying apparatus (step d)).

An embodiment in which the at least one conveying device is a component of a mixer has proven to be further advantageous.

In addition, a plurality of conveying devices that are optionally of different designs can be provided arranged in parallel or in series with one another.

Optionally, portions of the dried product are fed to the mixer before or during mixing (in step c)), whereby water fractions present in the fractions to be mixed can be bound. The resulting homogenous compound thus has a lower water content.

Preferably, the dried product is cooled after drying (step d)) or after grinding (step e)), which accelerates further processing and further use. Moreover, a chilled protein meal can be easily conditioned, for example, filled into bags since the cooling reduces the risk of moisture from the air condensing on the warm product.

In a further aspect, the invention also relates to a drying apparatus for use in at least one part of the aforementioned method, wherein the drying apparatus comprises at least one drying apparatus and a device for comminuting clumps of the homogenous compound.

The at least one drying apparatus comprises, for example, a thermal source and/or a radiation source for drying the homogenous compound.

The device for comminuting clumps of the homogenous compound may comprise elements acting on the homogenous compound or may be a special operating mode, for example of the drying apparatus itself or the parts thereof.

This drying apparatus is preferably designed as a fluidized bed dryer, wherein the movement of the fluidized bed and thus the specific operating mode of the fluidized bed dryer forms the device for comminuting clumps of the homogenous compound.

For example, a vibrating fluidized bed dryer is used, which ensures a simple comminution of clumps of the homogenous compound by means of the movement of the fluidized bed.

A shaking fluidized bed dryer, in which not only the fluidized bed moves but rather the whole fluidized bed dryer shakes, has proven to be particularly advantageous for comminuting clumps of the homogenous compound. In contrast to a vibrating fluidized bed dryer, a shaking fluidized bed dryer is more suitable for sticky materials. Furthermore, the transport of the homogenous compound or of the protein meal through the drying apparatus is improved as a result of a type of throwing movement which happens on the fluidized bed. In addition, due to this type of movement of the fluidized bed, drying takes place according to the “first-in”/“first-out” principle so that the portions of the homogenous compound, which are to be dried and which are introduced into the drying apparatus, are dried uniformly and continuously and thus not statically.

A movement of the fluidized bed or of the fluidized bed dryer with a low frequency and a high amplitude has proven to be very advantageous. The frequency of movement (also called shaking frequency) of the fluidized bed or of the shaking fluidized bed dryer is advantageously 2.5 Hz to 4.0 Hz. Ideally, the frequency of movement is 3.0 Hz to 3.5 Hz and particularly preferably 3.3 Hz to 3.4 Hz.

Alternatively, the drying apparatus is designed as a rotary dryer. In this context, a “rotary dryer” is understood to mean any drying apparatus having a rotating, advantageously mechanical device for comminuting clumps. Non-exhaustive examples include dryers with a grinding rotor system, paddle dryer, turbo dryer, etc.

Preferably, the temperature of the drying air supplied is less than or equal to 200° C., thereby facilitating sufficiently strong drying of the homogenous compound. A drying air temperature in the range from 110° C. to 160° C. has proven to be particularly preferred. Drying air temperatures in the aforementioned temperature ranges ensure sufficient drying of the homogenous compound without significantly reducing the quality thereof. The temperature of the homogenous compound is advantageously kept below 100° C. and particularly preferably below 70° C. during the drying process.

Preferably, at least two treatment zones are provided, which enables an advantageous treatment of the homogenous compound in the drying apparatus.

A drying apparatus having three treatment zones, namely a first treatment zone, a second treatment zone and a third treatment zone, has proven particularly advantageous. For example, the homogenous compound fed to the drying apparatus is pre-dried in the first treatment zone (drying zone), wherein advantageously the water content of the fed homogenous compound in particular is substantially reduced here. For example, the main drying of the fed homogenous compound, in which the water content of the homogenous compound is reduced to the desired extent, is carried out in the second treatment zone (drying zone). The third treatment zone (cooling zone) serves, for example, to cool the dried protein meal, as a result of which it is easier to handle, e.g., easier to pack.

Furthermore, it is also conceivable for the drying apparatus to have a plurality of further treatment zones. In this way, it is possible in some circumstances to achieve even more effective or more targeted drying of the homogenous compound and to take account of particular properties of the homogenous compound or of the protein meal to be produced.

Alternatively or additionally, two or more drying apparatuses can also be arranged one behind the other, so that a plurality of treatment zones are available for drying and/or cooling.

The invention is explained in more detail below with reference to an exemplary embodiment and a number of drawings. The drawings show:

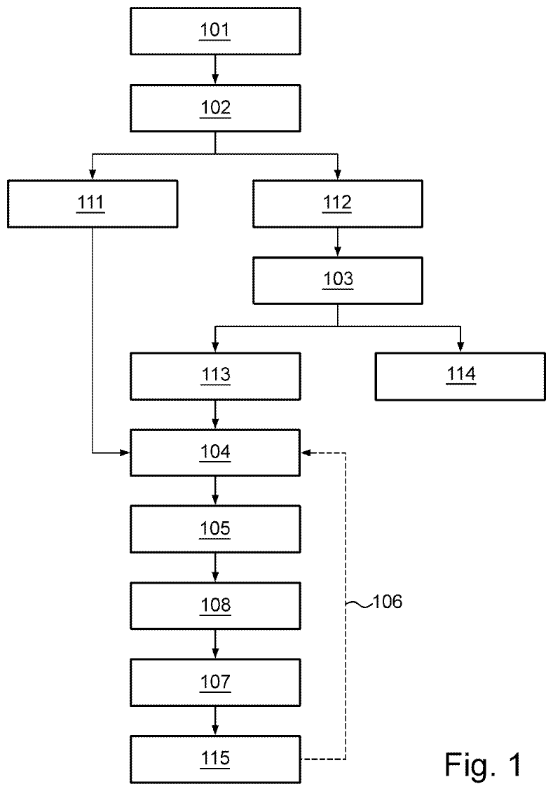

FIG. 1: a schematic representation of a method according to the invention;

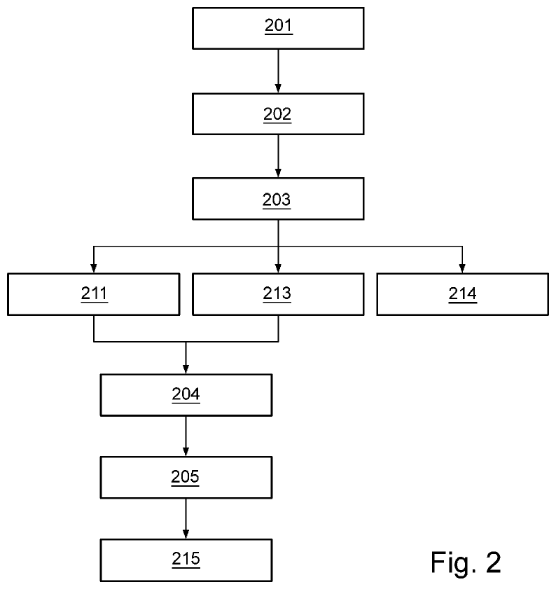

FIG. 2: a schematic representation of a further method according to the invention;

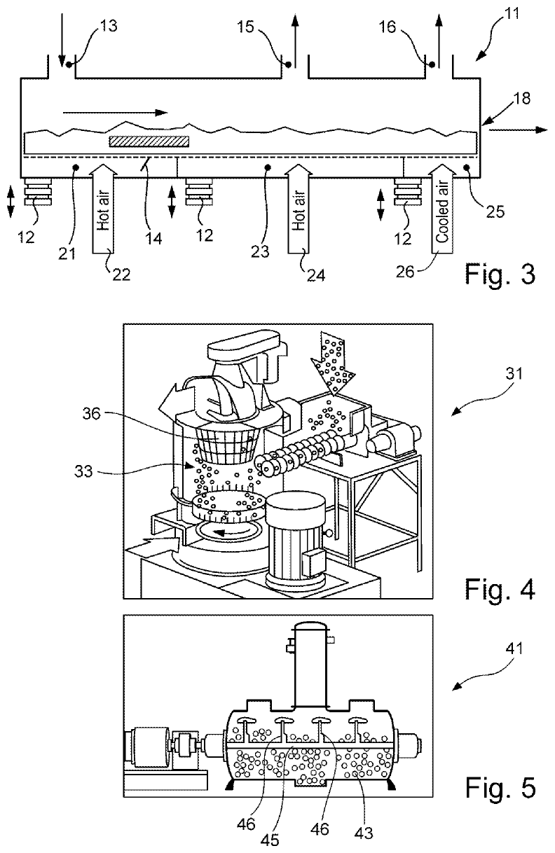

FIG. 3: a schematic side view of a drying apparatus according to the invention;

FIG. 4: a partial section of a variant of the drying apparatus shown in FIG. 3;

FIG. 5: a partial section of a further variant of the drying apparatus shown in FIG. 3.

According to the method shown in FIG. 1 for producing a protein meal from insect larvae, the insect larvae (here black soldier fly (BSF) larvae) are fed first (step 101). The insect larvae are crushed to form a pulp or purée in a press (step 102), wherein a solids-containing fraction 111 is separated from a liquid fraction 112.

Said liquid fraction 112 is split into an aqueous fraction 113 and a fat fraction 114 in a 2-phase separator. The water content of the aqueous fraction 113 can be reduced prior to further processing, for example in an evaporator or in a membrane filter.

The solids-containing fraction 111 and the aqueous fraction 113, which both contain protein, are mixed in a mixer to form a homogenous compound (step 104). Any clumps of the homogenous compound are comminuted during mixing. To reduce the water content of the homogenous compound, portions of the dried protein meal are fed to the mixer during mixing (step 106). Optionally, the homogenous compound is cooled after mixing.

The mixer has, for example, a conveying device by means of which the mixed homogenous compound is fed to the drying apparatus for drying. The conveying device is, for example, part of the mixer.

The homogenous compound is subsequently dried in a shaking fluidized bed dryer as drying apparatus (step 105) until the dried protein meal 115 is available. Any clumps of the homogenous compound are comminuted during drying. After or during drying (step 105), the dried protein meal 115 can be ground (step 107). The dried protein meal 115 is cooled after drying (step 105) (=step 108).

According to the alternative method shown in FIG. 2 for producing a protein meal from insect larvae, the insect larvae (here black soldier fly (BSF) larvae) are first fed to a cutter (step 201). In the cutter, the insect larvae are comminuted to form a pulp or a purée (step 202).

Said pulp or purée is split in a 3-phase separator (step 203) into a solids-containing fraction 211, an aqueous fraction 213 and a fat fraction 214. The water content of the aqueous fraction 213 can be reduced prior to further processing, for example in an evaporator or in a membrane filter.

The solids-containing fraction 211 and the aqueous fraction 213, which both contain protein, are mixed in a mixer to form a homogenous compound (step 204). Any clumps of the homogenous compound are comminuted during mixing. To reduce the water content of the homogenous compound, portions of the dried protein meal 215 can be fed to the mixer during mixing.

The homogenous compound is subsequently dried in a rotary mixer as drying apparatus (step 205) until the dried protein meal 215 is available.

For example, the dried protein meal 215 can then be ground, depending on the further use. Alternatively or additionally, the dried protein meal 215 can also be cooled.

The drying apparatus 11 shown in FIG. 3 is designed as a shaking fluidized bed dryer which is mounted on bearings 12. The drying apparatus 11 is also guided by means of the bearings 12, for example in the direction of the double arrows. The frequency of movement of the drying apparatus 11 is 2.5 Hz to 4.0 Hz, ideally 3.0 Hz to 3.5 Hz, preferably 3.3 Hz to 3.4 Hz.

As drying devices, the drying apparatus 11 comprises a first feed 22 for hot air having an air temperature of 140° C. to 150° C. and a second feed 24 for hot air having an air temperature of 150° C. to 180° C. or less than 200° C. and a third feed 26 for cooled air or ambient air having an air temperature of 15° C. to 25° C. The first feed 22 is provided in a first treatment zone 21, the second feed 24 is provided in a second treatment zone 23 and the third feed 26 is provided in a third treatment zone 25.

The drying apparatus 11 has an inlet opening 13 for the homogenous compound which, after being fed into the drying apparatus 11, comes to rest on the fluidized bed 14. Furthermore, two exhaust air openings 15 and 16 are provided through which air can escape from the drying apparatus 11.

The homogenous compound that comes to rest on the fluidized bed 14 is transported continuously by the movement of the fluidized bed 14, which movement is caused by the fluidized bed dryer that shakes during operation, from the inlet opening 13 to a discharge opening 18 from which the dried protein meal exits. During transport on the fluidized bed 14, the homogenous compound passes through the first treatment zone 21, in which the homogenous compound is pre-dried, the second treatment zone 23, in which the homogenous compound is dried to form the protein meal, and the third treatment zone 25, in which the dried protein meal is cooled.

The moving fluidized bed 14 forms the device for comminuting clumps of the homogenous compound of the drying apparatus 11.

FIG. 4 shows a drying apparatus 31 which is designed as a rotary dryer. The drying apparatus 31 has a grinding rotor system 36 in the drying chamber 33 as a device for comminuting clumps in the homogenous compound. The drying chamber 33 forms the drying device of the drying apparatus 31. The homogenous compound is ground during drying, and clumps which are present in the homogenous compound or which arise during the drying process are destroyed. The drying temperature in the drying chamber 33 is less than 200° C. The drying space 33 can be subdivided, for example, and have different temperatures in these subdivisions, so that the drying apparatus 31 has a plurality of treatment zones.

Instead of a grinding rotor system 36, a rotary mixer (not shown here) can be provided, which makes it easy to destroy clumps which are present in the homogenous compound or which form during the drying process. An advantageous geometry of the rotary mixer is selected as a function of the type of composition and/or the consistency of the homogenous compound.

FIG. 5 shows a drying apparatus 41 which is likewise designed as a rotary dryer, namely as a so-called turbo dryer. A drying chamber 43 forms the drying device of drying apparatus 41. In the drying chamber 43, a rapidly rotating rotor 45 with blades 46 is provided, which pushes the homogenous compound against the wall of the drying chamber 43 at high speed during drying, wherein clumps which are present in the homogenous compound or which are produced during the drying process are destroyed. The drying temperature in the drying chamber 43 is less than 200° C. The drying chamber 43 can also be subdivided and have different temperatures in these subdivisions, so that the drying apparatus 41 has a plurality of treatment zones.

### LIST OF REFERENCE SIGNS

11 Drying apparatus
12 Bearings
13 Inlet opening
14 Fluidized bed
15 First outlet opening
16 Second outlet opening
18 Discharge opening
21 First treatment zone
22 First feed
23 Second treatment zone
24 Second feed
25 Third treatment zone
26 Third feed
31 Drying apparatus
33 Drying chamber
36 Grinding rotor system
41 Drying apparatus
43 Drying chamber
45 Rotor
46 Blade
101 Feeding insect larvae
102 Comminuting insect larvae
103 Separating the pulp or purée
104 Mixing 111 and 113
105 Drying
106 Feeding 115 to 104
107 Grinding
108 Cooling
111 Solids-containing fraction
112 Liquid fraction
113 Aqueous fraction
114 Fat fraction
115 Dried protein meal
201 Feeding insect larvae
202 Comminuting insect larvae
203 Separating the pulp or purée
204 Mixing 211 and 213
205 Drying
211 Solids-containing fraction
213 Aqueous fraction
214 Fat fraction
215 Dried protein meal

1. A method for producing a protein meal from insects, in particular from insect larvae and from insect pupae, or from worms, comprising the steps of:
a) comminuting the insects or worms to form a pulp or purée;
b) splitting the pulp or the purée into a fat fraction, a solids-containing fraction and an aqueous fraction;
c) mixing the aqueous fraction and the solids-containing fraction to form a homogenous compound;
d) drying the homogenous compound, with a dried protein meal being obtained.

2. The method according to claim 1,
wherein the water content of the aqueous fraction is reduced before step c).

3. The method according to claim 2,
wherein the water content of the aqueous fraction is reduced in an evaporator or in a filter, advantageously in a membrane filter.

4. The method according to claim 1, wherein the dried protein meal is ground after step d).

5. The method according to claim 1, wherein the comminution (step a); is carried out in a cutter or in a press.

6. The method according to claim 1,
wherein the splitting of the pulp or purée according to step b) takes place in a separator, advantageously in a 2- or 3-phase separator.

7. The method according to claim 1, further comprising the step e) for comminuting clumps of the homogenous compound in step c) or in step d).

8. The method according to claim 1,
wherein the homogenous compound is fed to the drying apparatus for drying during and/or after step c) by means of at least one conveying device, wherein the at least one conveying device is advantageously part of a mixer.

9. The method according to claim 1,
wherein portions of the dried protein meal are fed to the mixer (in step c).

10. The method according to claim 1, wherein the dried protein meal is cooled after step d) or after step e).

11. A drying apparatus for use in a method according to claim 1, comprising at least one drying apparatus, characterized in that
a device is provided for comminuting clumps of the homogenous compound.

12. The drying apparatus according to claim 11, designed as a fluidized bed dryer, preferably as a shaking fluidized bed dryer having a frequency of movement advantageously at 2.5 Hz to 4.0 Hz, ideally at 3.0 Hz to 3.5 Hz, preferably at 3.3 Hz to 3.4 Hz.

13. The drying apparatus according to claim 11, designed as a rotary dryer.

14. The drying apparatus according to claim 11, wherein the temperature of the drying air supplied is less than or equal to 200° C., preferably 110° C. to 160° C.

15. The drying apparatus according to claim 11,
wherein at least two treatment zones are provided.
